(19) United States
(12) Patent Application Publication
(10) Pub. No.: US 2025/0270833 A1
(43) Pub. Date: Aug. 28, 2025

(54) THE DIP DOSAGE CALCULATOR

(71) Applicant: The Dip App, Inc., San Jose, CA (US)

(72) Inventors: Michael Hicks, San Jose, CA (US); Robert Day, La Honda, CA (US)

(21) Appl. No.: 18/587,392

(22) Filed: Feb. 26, 2024

(51) Int. Cl. E04H 4/14; C02F 1/68; C02F 103/42; G06Q 10/20

(52) U.S. Cl. CPC E04H 4/14; C02F 1/68; G06Q 10/20; C02F 2103/42; C02F 2209/006

(57) ABSTRACT

This disclosure provides systems, devices, apparatus, and methods, including computer programs encoded on storage media, for processing pool care information. A processing device receives an input indicating a condition associated with water of a swimming pool. The condition has an impact on balancing a water chemistry of the water. The processing device computes, based on the input, a current water chemistry of the water of the swimming pool and determines a target water chemistry for the water of the swimming pool. The target water chemistry is different from the current water chemistry. The processing device outputs a pool care recommendation for the balancing the water chemistry of the water from the current water chemistry to the target water chemistry. The pool care recommendation is based on how the condition impacts the balancing the water chemistry.

### TECHNICAL FIELD

The present disclosure relates generally to software applications, such as mobile software applications (Apps), and more particularly, to indicating water chemistry information for swimming pools.

### BACKGROUND

Swimming pools require regular maintenance and servicing to keep the water within a pool clear and attractive to swimmers. For example, if a balanced water chemistry is not maintained for the pool water, the water may become cloudy and less inviting to the swimmers. Unfortunately, not all pool owners, or oftentimes even lay workers of a pool service company, possess sufficient knowledge to engage in skilled pool care. Such individuals may resort to trial-and-error techniques or guesswork approaches when attempting to provide a balanced water chemistry for the swimming pool. Further, the pool care persons may have no way of knowing whether the approach they have implemented has achieved the optimum water chemistry or not. Accordingly, there is a need to provide efficient and simplified pool care information to both pool owners and pool service professionals.

### BRIEF SUMMARY

The following presents a simplified summary of one or more aspects in order to provide a basic understanding of such aspects. This summary is not an extensive overview of all contemplated aspects. This summary neither identifies key or critical elements of all aspects nor delineates the scope of any or all aspects. Its sole purpose is to present some concepts of one or more aspects in a simplified form as a prelude to the more detailed description that is presented later.

In aspects of the disclosure, a system, a method, a computer-readable medium, and an apparatus are provided. The method includes receiving an input indicating a condition associated with water of a swimming pool, the condition having an impact on balancing a water chemistry of the water; computing, based on the input, a current water chemistry of the water of the swimming pool; determining a target water chemistry for the water of the swimming pool, the target water chemistry being different from the current water chemistry; and outputting, by a processing device, a pool care recommendation for the balancing the water chemistry of the water from the current water chemistry to the target water chemistry, the pool care recommendation being based on how the condition impacts the balancing the water chemistry.

To the accomplishment of the foregoing and related ends, the one or more aspects comprise the features hereinafter fully described and particularly pointed out in the claims. The following description and the drawings set forth in detail certain illustrative features of the one or more aspects. These features are indicative, however, of but a few of the various ways in which the principles of various aspects may be employed.

### DESCRIPTION OF THE DRAWINGS

FIG. 1 is a diagram illustrating an example content generation system.

FIG. 2 is a diagram illustrating a dosage calculation application being launched for a pool care application.

FIG. 3 is a diagram illustrating an execution of the dosage calculation application based on an input water chemistry.

FIG. 4 illustrates a diagram in which a target Langelier saturation index (LSI) is manipulated relative to an LSI reading based on one or more adjustments to the water chemistry.

FIG. 5 is a diagram illustrating a dosage recommendation generated based on input values to the dosage calculation application.

FIG. 6 is a diagram illustrating a chemical preference feature of the pool care application.

FIG. 7 is a block diagram illustrating a system architecture for generating a pool care recommendation.

FIG. 8 is a flowchart of a method of processing pool care information.

FIG. 9 is a high-level illustration of an exemplary computing device that can be used in accordance with the systems and methodologies disclosed herein.

### DETAILED DESCRIPTION

The detailed description set forth below in connection with the drawings describes various configurations and does not represent the only configurations in which the concepts described herein may be practiced. The detailed description includes specific details for the purpose of providing a thorough understanding of various concepts. However, these concepts may be practiced without these specific details. In some instances, well known structures and components are shown in block diagram form in order to avoid obscuring such concepts.

Several aspects are presented with reference to various apparatus and methods. These apparatus and methods are described in the following detailed description and illustrated in the accompanying drawings by various blocks, components, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip, baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software, whether referred to as software, firmware, middleware, microcode, hardware description language, or otherwise, shall be construed broadly to mean instructions, instruction sets, code, code segments, program code, programs, subprograms, software components, applications, software applications, software packages, routines, subroutines, objects, executables, threads of execution, procedures, functions, or any combination thereof.

Accordingly, in one or more example aspects, implementations, and/or use cases, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, such computer-readable media can comprise a random-access memory (RAM), a read-only memory (ROM), an electrically erasable programmable ROM (EEPROM), optical disk storage, magnetic disk storage, other magnetic storage devices, combinations of such types of computer-readable media, or any other medium that can be used to store computer executable code in the form of instructions or data structures that can be accessed by a computer.

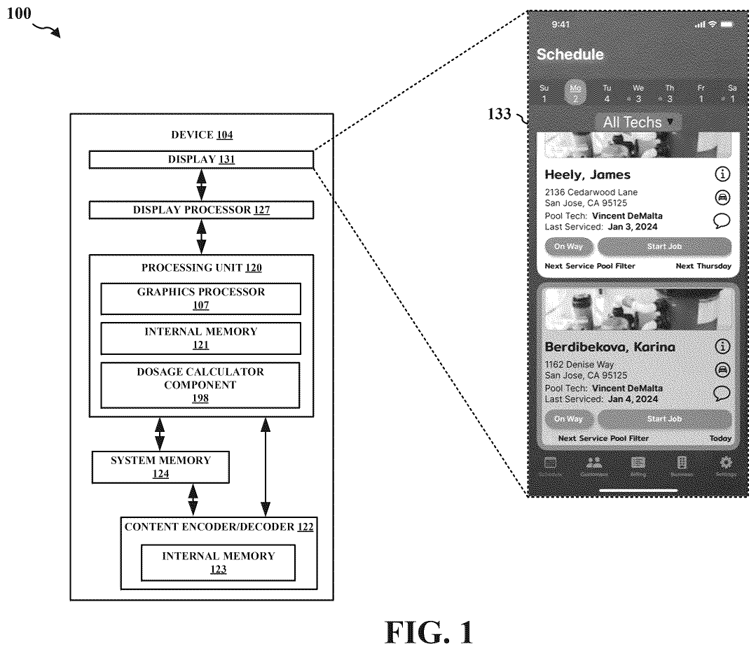

FIG. 1 is a block diagram that illustrates an example content generation system 100. The content generation system 100 includes a device 104 that has one or more components or circuits for performing various functions described herein. The device 104 may include a display 131, a display processor 127, a processing unit 120, a system memory 124, a content encoder/decoder 122, etc. In some examples, the device 104 is a smartphone configured to execute mobile software applications (apps). The display 131 may provide a user interface (UI) 133 to the software application(s) executing on the device 104. For example, a user may interact with graphical content associated with an output of a software application through the UI 133 provide provided via the display 131. In other examples, the graphical content may be communicated to another device for display.

The processing unit 120 may include a graphics processor 107 and an internal memory 121. The processing unit 120 may be configured to perform graphics processing using the graphics processor 107 (e.g., based on a graphics processing pipeline). The processing unit 120 may also generate the graphical content displayed through the UI 133. The processing unit 120 further includes a dosage calculator component 198, as will be discussed in further detail below, for performing various aspects and functionality described herein.

The display processor 127 may be configured to perform one or more display processing techniques on one or more frames/graphical content generated by the processing unit 120 before the frames/graphical content is displayed via the display 131 and UI 133. While the example content generation system 100 illustrates a display processor 127, it should be understood that the display processor 127 is one example of a processor that can perform the functions descried herein and that other types of processors, controllers, etc., may be used as a substitute for the display processor 127. The display 131 may be configured to display or otherwise present graphical content processed/output by the display processor 127. In some examples, the display 131 may be a liquid crystal display (LCD), a plasma display, an organic light emitting diode (OLED) display, a projection display device, or any other type of display device.

Memory external to the processing unit 120 and the content encoder/decoder 122, such as system memory 124, may be accessible to the processing unit 120 and the content encoder/decoder 122. For example, the processing unit 120 and the content encoder/decoder 122 may be configured to read from and/or write to external memory, such as the system memory 124. The processing unit 120 includes the internal memory 121. The content encoder/decoder 122 may also include an internal memory 123. The processing unit 120 and the content encoder/decoder 122 may be communicatively coupled to the system memory 124 over a bus. In some examples, the processing unit 120 and the content encoder/decoder 122 may be communicatively coupled to the internal memories 121/123 over the bus or via a different connection. The content encoder/decoder 122 may be configured to receive graphical content from any source, such as the system memory 124 and/or the processing unit 120 and encode or decode the graphical content. In some examples, the graphical content may be in the form of encoded or decoded pixel data. The system memory 124 may be configured to store the graphical content in an encoded or decoded form.

The internal memories 121/123 and/or the system memory 124 may include one or more volatile or non-volatile memories or storage devices. In some examples, the internal memories 121/123 or the system memory 124 may include random-access memory RAM, static random-access memory (SRAM), dynamic random-access memory (DRAM), erasable programmable ROM (EPROM), EEPROM, flash memory, a magnetic data media, optical storage media, or any other type of memory. The internal memories 121/123 and the system memory 124 are a non-transitory storage medium according to some examples. The term “non-transitory” may indicate that the storage medium is not embodied in a carrier wave or a propagated signal. However, the term “non-transitory” should not be interpreted to mean that the internal memories 121/123 or the system memory 124 is non-movable or that its contents are static. As one example, the system memory 124 may be removed from the device 104 and moved to another device. As another example, the system memory 124 may not be removable from the device 104.

The processing unit 120 may be a central processing unit (CPU), a graphics processing unit (GPU), or any other processing unit configured to provide content for display. The content encoder/decoder 122 may be any processor configured to perform content encoding and content decoding. In some examples, the processing unit 120 and/or the content encoder/decoder 122 may be integrated into a motherboard of the device 104. The processing unit 120 may be present on a graphics card that is installed in a port of the motherboard of the device 104 or may be otherwise incorporated within a peripheral device configured to interoperate with the device 104. The processing unit 120 and/or the content encoder/decoder 122 may include one or more processors, such as one or more microprocessors, GPUs, application specific integrated circuits (ASICs), field programmable gate arrays (FPGAs), arithmetic logic units (ALUs), digital signal processors (DSPs), discrete logic, software, hardware, firmware, other equivalent integrated or discrete logic circuitry, or any combination thereof. If the techniques are implemented partially in software, the processing unit 120 and/or the content encoder/decoder 122 may store instructions for the software in a suitable, non-transitory computer-readable storage medium (e.g., memory) and may execute the instructions in hardware using one or more processors to perform the techniques of this disclosure. Any of the foregoing, including hardware, software, a combination of hardware and software, etc., may be considered to be one or more processors.

In certain aspects, the processing unit 120 (e.g., CPU, GPU, etc.) may include a dosage calculator component 198, which may include software, hardware, or a combination thereof for receiving an input indicating a condition associated with water of a swimming pool, the condition having an impact on balancing a water chemistry of the water; computing, based on the input, a current water chemistry of the water of the swimming pool; determining a target water chemistry for the water of the swimming pool, the target water chemistry being different from the current water chemistry; and outputting, by a processing device, a pool care recommendation for the balancing the water chemistry of the water from the current water chemistry to the target water chemistry, the pool care recommendation being based on how the condition impacts the balancing the water chemistry. Although the following description may be focused on processing pool care information, the concepts described herein may be applicable to other similar processing techniques.

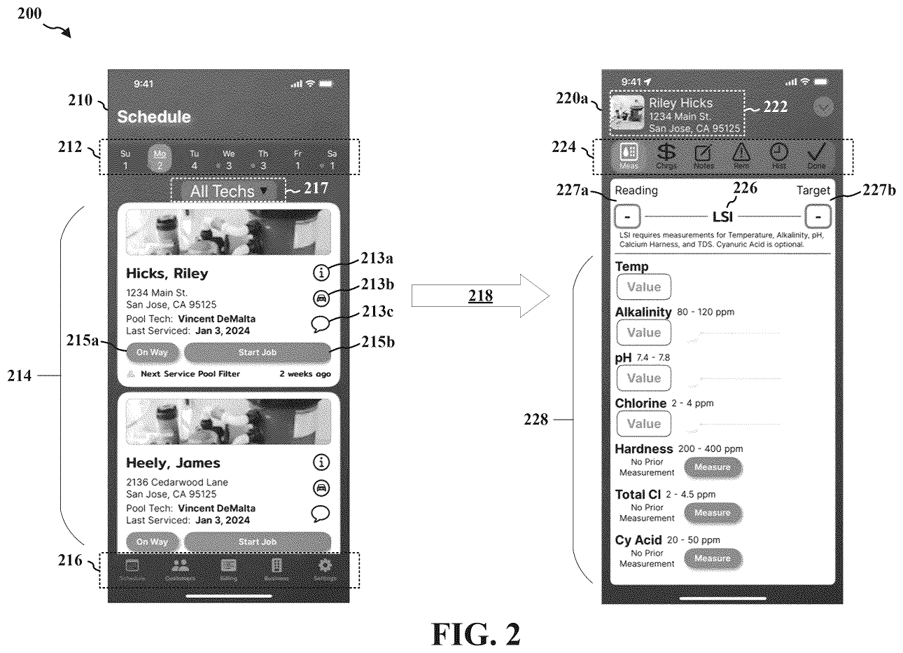

FIG. 2 is a diagram 200 illustrating a dosage calculation application 220a being launched 218 for a specific (pool) identifier (ID) of a pool care application 210. The dosage calculation application 220a generates recommendations to facilitate managing the water chemistry of one or more swimming pools associated with the pool care application 210. For example, a pool service processional who cares for multiple swimming pools may input and/or view information, and receive recommendations, for multiple clients via the pool care application 210. Similarly, a pool owner may self-care for their own swimming pool based on inputs and/or information, such as recommendations, generated and provided via the pool care application 210. Each client or pool owner may be associated with a different ID which may, for example, correspond to a client ID, user ID, pool ID, or the like. When the dosage calculation application 220a is launched 218, the inputs and outputs of the dosage calculation application 220a are specific to the respective swimming pools (i.e., IDs) being managed via the pool care application 210.

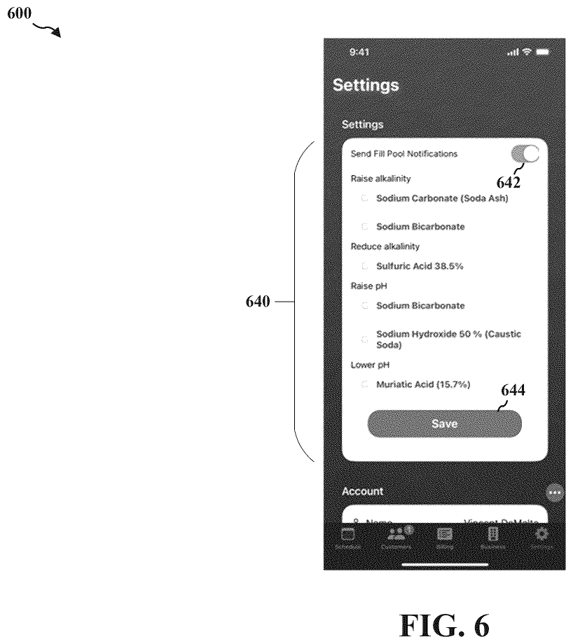

The pool care application 210 includes various functionality and features 216, such as functionality and features 216 related to schedules, customers, billing, business, settings, etc. The customers feature initiates and displays customer information associated with the pool care application 210. For example, a pool care professional may have different customer information that the pool care professional needs to maintain via the customers feature. The billing feature initiates and displays billing information related to pool care services provided via a pool person, such as a pool care professional using the pool care application 210. Similarly, the pool care professional may have various business information that the pool care professional needs to maintain, via the business feature, regarding the business of the pool care professional. The settings feature provides access to basic and advanced settings of the pool care application 210. The settings feature is further referenced below with respect to FIG. 6, where chemicals that are being carried on a truck of a pool service professional can be entered/indicated as a basis for the recommendation being generated by the pool care application 210.

The diagram 200 illustrates a user interface associated with the schedule feature of the pool care application 210. For example, the pool care application 210 receives an input to display schedule information and generates a user interface indicating past, present, and/or future schedule information. Although the diagram 200 illustrates a calendar ribbon 212 and a list of associated pool owner “cards” 214, it is understood that the schedule information can be displayed and/or configured in any number of ways.

The calendar ribbon 212 provides quick and efficient access to past, present, and/or future pool care information and tasks. For example, the pool care application 210 may receive input via the calendar ribbon 212 to display schedule information for Monday the 2nd. Responsive to receiving the input, the pool care application 210 generates one or more cards 214 associated with pool care tasks corresponding to the indicated date. In an example, the cards 214 may display respective information for pool owners associated with the pool care application 210, such as a pool owner name, a pool owner address, a pool technician assigned to the pool owner, and/or a date of last service. The pool care application 212 may include a drop-down menu or other feature to filter the cards 214 based on certain criteria. For example, the filter 217 may be selectable to display the cards 214 associated with all, or a subset of, pool technicians for a pool service company. Other information displayed via the cards 214 may include one or more indicators of a next pool service activity and when the next pool service activity is to be, or should have already been, performed.

Each card 214 includes one or more selectable sub-features 213a-213c. For example, a first sub-feature is an information sub-feature 213a for identifying basic information about the pool owner, pool information, service information, and the like. A second sub-feature is a tracking and/or route information sub-feature 213b that provides tracking and/or route information to a client pool owner. That is, when the pool care professional is in route to a client destination, a client pool owner who also has access to the pool care application 210 may receive real-time location information of the pool care professional. A third sub-feature is a messaging sub-feature 213c, where the pool care professional and the client pool owner may communicate back and forth with each other with real-time (text) messages via the pool care application 210.

Each card 214 may also include one or more buttons 215a-215b. For example, the pool care professional may notify the client pool owner that the pool care professional is in route to the client destination by selecting an “on way” button 215a associated with the card 214 of the client pool owner. Responsive to receiving input through the on way button 215a, a real-time notification is transmitted to a second device (e.g., a smartphone of the client pool owner) to indicate that the pool service professional is in route to the client destination. The real-time notification may be received at the second device via the pool service application 210 running on the second device, or through other means of indication, such as through a general text message notification.

Other types of notifications that may be received at the second device can include a notification that indicates when the pool service is complete, a notification that indicates when it is safe for a swimmer to get back into the water, etc. For pool owners who self-care, not every pool owner has sufficient knowledge to engage in skilled pool care. Thus, further types of notifications may include indications of when it is time to check or service the pool again. For instance, an example notification my indicate that the pool was last serviced 7 days ago and that it is now time to perform X. Notifications and/or recommendations may be based on historical data. The historical data may be for the same pool being serviced as well as for other pools of similar characteristics. Based on the historical data, the pool care application can perform forecasting to indicate to pool persons a recommended way to care for the pool.

Some pool care recommendations may be based on predictive modeling, such as through artificial intelligence (AI) or machine learning (ML) models. Once the pool care application 210 receives enough data for how pools are cared for (e.g., via historical conditions), the system can use predictive models to provide pool care suggestions based on the data. The data can be shared across pool owners with varying pool and environment conditions to implement the predictive models. For example, a predictive model may provide different suggestions to pool owners having pools in different climates based on how various pool conditions have historically reacted to different temperatures/climates. Thus, instead of the pool care application 210 outputting a basic reminder to service a pool, the output can include predictive information that is catered to specific conditions of the pool and/or environment. In further examples, the pool care application 210 can use as input how much chemical a pool owner still has remaining (e.g., based on historical dose logs) and generate a notification when the chemical is running low or when it is time to buy more of the chemical as well as an indication of where/how to by the chemical.

Once the pool care professional arrives at the client pool location, the pool care application 210 may be used for servicing the client pool. Each card 214 includes a “start job” button 215b. Responsive to receiving input through the start job button 215b, the pool care application 210 launches 218 the dosage calculation application 220. Each card 214 may be linked to the ID associated with the pool owner whose job is being started 215b. Thus, the dosage calculation application 220 can provide a pool-specific output for the pool associated with the ID. In other examples, the dosage calculation application 220a may provide general outputs that are typically applicable to most pools.

The dosage calculation application 220a (e.g., the dip dosage calculator) may be implemented on devices of pool service professionals and/or devices of pool owners, including self-care pool owners and client pool owners. Given that the dosage calculation application 220a may be pool-specific, the user interface may include header information 222 indicating the pool owner (e.g., ID) for which dosage calculation application 220a has been launched 218. The head information 222 may include a subset, or all, of the pool owner information displayed on the card 214 used to launch 218 dosage calculation application 220a.

Similar to the user interface of the pool care application 210, the user interface of the dosage calculation application 220a also includes various functionality and features 224, such as functionality and features 224 related to measurements, charges, notes, remediation, history, done/complete, etc. The charges feature displays customer charges related to the pool care. The notes feature initiates and displays custom/editable notes related to the pool care. The remediation feature initiates and displays corrective actions that are being recommended for the pool. The history feature initiates and displays historical data for the pool, conditions, and/or environment associated with the pool. The done/complete feature closes the dosage calculation application 220a and causes the device to re-display a user interface of the pool care application 210.

The dosage calculation application 220a can have different implementations based on a chemical composition of the water, client preferences, client behavior, internal/external conditions to the water, etc. For example, the dosage calculation application 220a may utilize data/intelligence to output a recommendation of a type of chemical that should be used to balance the water chemistry (of a specific swimming pool) back to an industry standard level. The recommendation may also include other information, such as an amount of the recommended chemical that is needed to balance the water chemistry.

Another example output of the dosage calculation application 220a is a Langelier saturation index (LSI) 226. In water chemistry, the LSI 226 is a measure of the ability of the water to absorb and hold solids in a solution, and is specific to the calcium content of the water. A scale on which the LSI 226 is measured can be narrow. Thus, a small change in the water chemistry can result in a significant difference in the pool water. That is, to change a current LSI reading field 227a in view of a target LSI reading field 227b, a (small) adjustment may be needed to one or more of the value fields 228, which may be used to provide the LSI 226.

To output a value for the current LSI reading field 227a for the water, the dosage calculation application 220a may require a plurality of values for the input value fields 228. The input value fields 228 include temperature, alkalinity, potential hydrogen (pH), chlorine, calcium hardness, total dissolved solids (TDS) and, optionally, cyanuric acid. The dosage calculation application 220a is capable of receiving an input temperature value in Fahrenheit, Celsius, or Kelvin, such as to output a value for the LSI reading field 227a. In an example, the alkalinity value may range from 80-120 parts per million (ppm), the pH value may range from 7.4-7.8, the chlorine value may range from 2-4 ppm, the hardness may range from 200-400 ppm, a total chlorine value may range from 2-4.5 ppm, and the cyanuric acid value may range from 20-50 ppm.

The input values for the input value fields 228 can be input to the dosage calculation application 220a by direct manual input of one or more measurement values. In some examples, the values for the input value fields 228 are provide from historical/prior measurement values. In other examples, historical/prior measurement values are not available for certain input value fields 228, such that a measurement is needed to provide an input for the dosage calculation application 228.

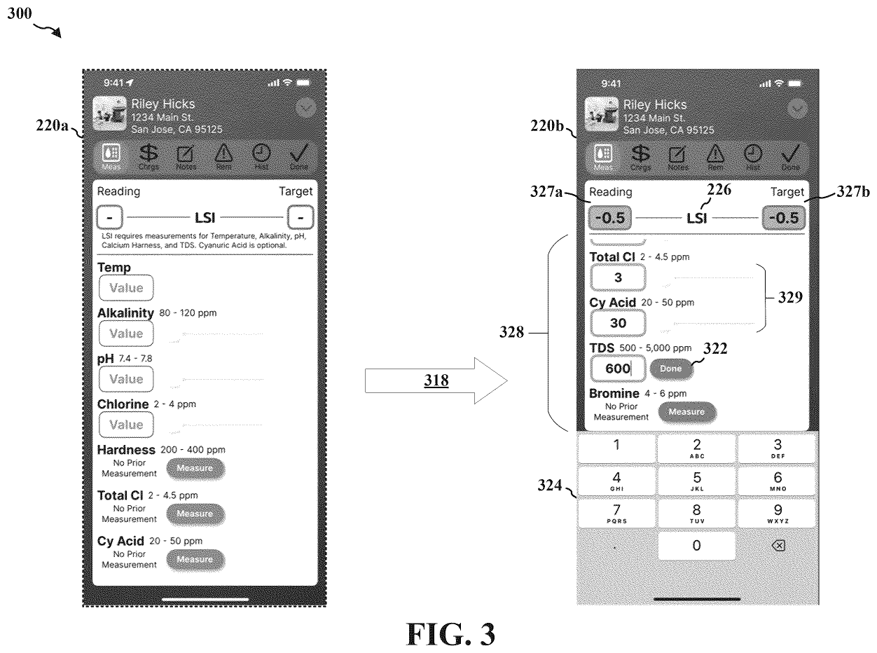

FIG. 3 is a diagram 300 illustrating an execution 318 of the dosage calculation application 220a-220b based on input values 328. For ease of reference, the dosage calculation application 220a in the diagram 300 is similar to the dosage calculation application 220a in the diagram 200.

To execute 318 the dosage calculation application 220b, values 328 are input to the value fields of the dosage calculation application 220b. For manual input of the values 328, the values 328 may be received by the dosage calculation application 220b via a keypad 324 or any other suitable means of input. The input values 328 are within the range associated with each respective input value field. For example, total chlorine=3 is within the range of 2-4.5 ppm, cyanuric acid=30 is within the range of 20-50 ppm, and TDS=600 is within the range of 500-5,000 ppm. In some examples, input value fields, such as for Bromine having a range of 4-6 ppm, may not have historical/prior measurement values available and may need to be measured to provide a corresponding input value 328.

After the input values 328 are entered into an input value field, a “done” button 322 is selected that causes the dosage calculation application 220b to update. For example, the LSI 226 may update based on the chemistry information. The diagram 300 indicates that the actual LSI reading 327a is −0.5. The target LSI 327b may default to the same value of −0.5 as the actual LSI reading 327a until a sliding scale feature 329 is manipulated. That is, a button on the sliding scale feature 329 may be slid left or right to independently adjust the target LSI 327b separate from the LSI reading 327a. In some examples, when the button on the sliding scale feature 329 is slid/positioned at its farthest point to the left, the target LSI 327b and the LSI reading 327a output the same value.

When the dosage calculation application 220b is executed 318 based on the chemistry information, one or more values may be logged/stored in a database. That is, the input values 328 for input value fields, such as temperature, alkalinity, pH, chlorine, calcium hardness, TDS, cyanuric acid, bromine, etc., are saved/stored in the database to create a historical record that the pool care application can refer back to when generating future notifications and recommendations. Historical reports/records may also be communicated to a pool owner device via the pool care application 210, or by other means, for the pool owner to view the data.

The dosage calculation application 220b can indicate the LSI 226 based on the input values 328 to the input value fields. That is, in the example of diagram 300, the dosage calculation application 220b output the actual LSI reading 327a and the target LSI 327b based on the on the input values 328 to the input value fields. The dosage calculation application outputs the same value (e.g., −0.5) for the LSI reading 327a and the target LSI 327b when the sliding button of the sliding bar feature 329 is located at a left most position of the sliding bar. In other words, when the sliding button is at the leftmost position of the sliding bar feature 329, an algorithm used to generate the target LSI 327b takes as input for the LSI calculation the same input value 328 as used by an algorithm to generate the LSI reading 327a, which ultimately results in the same output being generated for both the LSI reading 327a and the target LSI 327b.

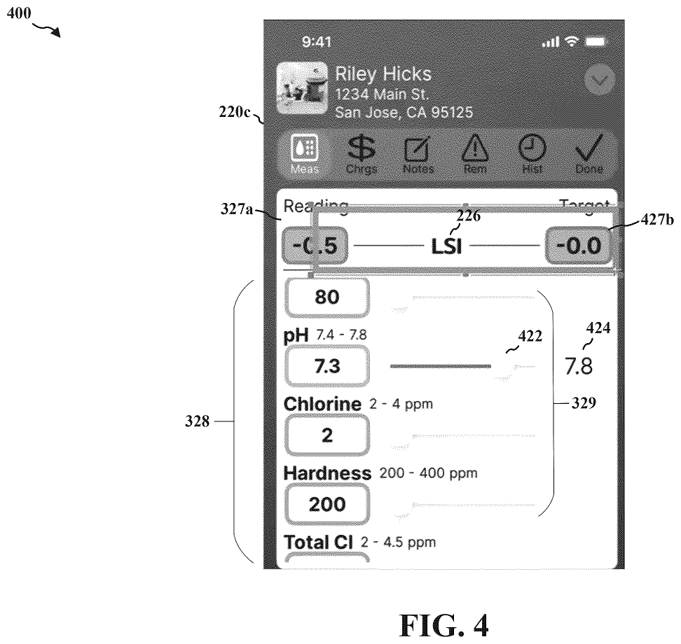

FIG. 4 illustrates a diagram 400 in which the target LSI 427b is manipulated relative to the LSI reading 327a based on one or more adjustments to the water chemistry values. For example, a desirable target LSI 427b may be 0.0, despite the current LSI reading 327a indicating that the LSI 226 is presently equal to −0.5 based on the input values 328 to the input value fields.

The target LSI 427b is manipulated in the dosage calculation application 220c by sliding the button(s) 422 along one or more bars of the sliding bar feature 329. For example, the button 422 corresponding to the pH level is shifted to the right to test what would happen to the LSI 226 if the pH is increased. In the example of diagram 400, repositioned button 422 along the sliding scale feature 329 provides a modified pH value 424 of 7.8. The dosage calculation application 220c is re-executed based on the modified pH value 424 to output an updated target LSI 427b of 0.0. In the example, the updated target LSI 427b results from changing the pH value while leaving the other input values 328 of the water chemistry unchanged. In other examples, any combination of sliding the various button(s) 422 to different degrees along on the sliding bar feature 329 cause the target LSI 427b to be updated relative to the LSI reading 327a, in some cases achieving the same desired target LSI 427b of 0.0. For instance, if the button 422 for the pH level is dropped to a value of 4.6, but another button associated with a different value is increased, the net effect may still be that the target LSI 427b is equal to 0.0.

After the chemical readings are input to the dosage calculation application 220c, the user interface may receive input that causes the pool care application to output a recommendation of a chemical type and a quantity of the chemical type to add to the water for balancing the water chemistry. For example, the pool care application uses, as input, chemical measurements (e.g., current measurements and/or historical data points) to output the recommendation. The values are stored in a database and combined with information from an application program interface (API) server, such as what the weather is going to be like for the next few days, or other metrics, to output the recommendation/action for dosing a specific swimming pool. The output of the LSI calculation may be used as one of the inputs that the system uses to generate the recommendation.

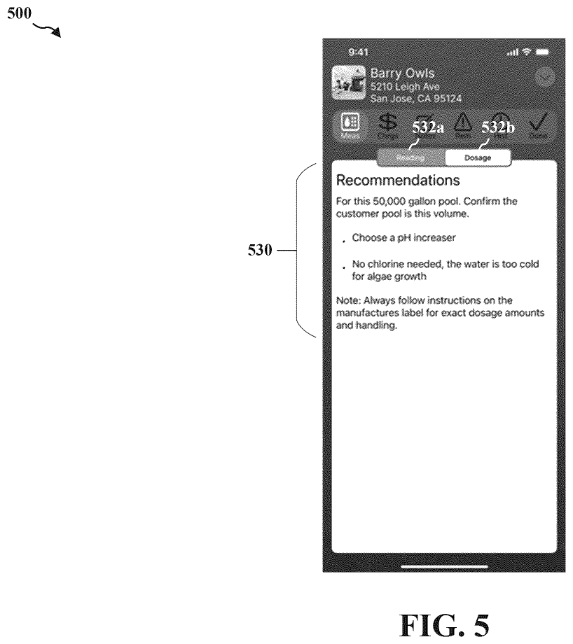

FIG. 5 is a diagram 500 illustrating a dosage recommendation 530 that is generated based on input values to the dosage calculation application. The input values correspond to a reading tab 532a associated with the dosage calculation/recommendation, whereas the dosage recommendation 530 itself corresponds to a dosage tab 532b. For example, the dosage calculation application may include a “request dosage” button that uses as input the information associated with the reading tab 532a to generate a recommendation 530 on the dosage tab 532b.

The dosage calculation application may log the readings/value(s) that are input to the input value fields of the dosage calculation application. Using the input values, such as input temperature values, the dosage calculation application may generate the recommendation 530 based on not only the current conditions of a specific swimming pool, but also based on ambient conditions associated with the swimming pool, to generate a pool-specific recommendation 530. For example, the recommendation may be to pre-condition the water a certain way when there is expected to be a temperature change from 51 degrees Fahrenheit to 54 degrees Fahrenheit. If the weather forecast indicates that the temperature is going to be 3 degrees warmer in the next 3 days, the pool may have to be treated differently than if the temperature were not going to change over the next 3 days. Inexperienced pool cleaners are oftentimes “chasing the tail” when weather conditions are not accounted for. That is, such pool cleaners are reactive to changes in the water chemistry resulting from changed weather conditions, rather than being proactive in view of upcoming weather forecasts.

Algae does not tend to grow if the water temperature is 52 degrees or below. Thus, chlorine does not need to be added to the pool water at such temperatures. However, an inexperienced pool cleaner may observe that there is no chlorine in a pool that is below 52 degrees and may add chlorine to the water anyway. This would not only result in unnecessary chemical waste, but could also throw off other parts of the water chemistry. Therefore, a generated recommendation 530 by the dosage calculation application may be based on different factors, such as when water temperate is 50 degrees as opposed to 65 degrees.

The dosage calculation application can also generate recommendations based on degree-level granularity, such as a change in the water temperature from 65 degrees to 66 degrees. In further examples, the dosage calculation application generates recommendations 530 based on temperature ranges, such as ranges for cold water, intermediate water, and/or hot water, as there may be a change in the water behavior among the different temperature ranges. This change may affect how much chlorine is added to the water, the way in which the pH is addressed, etc. For example, there may be a large change in pH when there is no change in water temperature kind. Thus, the generated recommendation 530 may be based on the water being in the ‘cold water’ temperature range and that the temperature is expected to change over the next 3 days.

In the diagram 500, the generated recommendation 530 is for a 50,000 gallon swimming pool. The recommendation 530 indicates to confirm that the customer pool is of this volume of water. The treatment of a leaky pool may be different than the treatment of a non-leaky pool, so if the pool is not of the indicated volume of water, the recommendation 530 may change. For example, the recommendation 530 may indicate that liquid chlorine is to be used for non-leaky pools, but chlorine tablets are to be used for leaky pools, such as to address cyanuric acid levels. If the volume of the pool associated with the recommendation 530 is confirmed to be 50,000 gallons, the generated recommendation 530 may describe further courses of action. For example, the generated recommendation 530 indicates to choose a pH increase and that no chlorine needs to be added to the water because the water is too cold for algae growth.

While the recommendation 530 in the diagram 500 notes to always follow instructions on the manufacturers label for exact dosage amounts and handling, the recommendation 530 itself can also be generated based on supplemental data. For example, the instructions on the back of a chemical container may provide sub-optimal outcomes if other factors, such as the weather, are not taken into account. Thus, dosage calculation application may account for weather conditions, leaky pool conditions, etc., when generating the recommendation 530.

FIG. 6 is a diagram 600 illustrating a chemical preference feature 640 of the pool care application. The chemical preference feature 640 can be within the settings feature described above with respect to FIG. 2.

The pool care application may allow a pool service professional to indicate which chemicals are currently being carried on the truck of the pool service professional and/or a chemical preference for treating a pool, so that the dosage calculation application can generate a recommendation based on the indicated chemicals and/or the chemical preference. For example, the chemical preference feature 640 may receive input that the pool care person has/prefers sodium carbonate (soda ash) and/or sodium bicarbonate if the alkalinity is to be raised. The chemical preference feature 640 may receive input that the pool care person has/prefers sulfuric acid (38.5%) if the alkalinity is to be reduced. Similarly, the chemical preference feature 640 may receive input that the pool care person has/prefers sodium bicarbonate and/or sodium hydroxide 50% (caustic soda) if the pH is to be raised. The chemical preference feature 640 may receive input that the pool care person has/prefers muriatic acid (15.7%) if the pH is to be reduced. The chemical preference feature 640 may further include a “send fill pool notifications” option 642 that can be toggled on or off to indicate when water should be added to the pool. After the preferences have been selected, a save button 644 is executed via the chemical preferences feature 640 to save/store the chemical preferences.

The generated recommendation can also vary based on internal and/or external conditions, even in view of a same set of indicated preferences. For example, if the cost of water is higher in California than in Mississippi, the generated recommendation may be to add a chemical to a swimming pool located in California versus simply adding more water to a swimming pool located in Mississippi, even when the pools are associated with the same or similar water chemistries and other environmental conditions. The generated recommendation may account for chemicals that are “in stock” on a pool professional's truck, such that the recommendation may indicate an optimal way to treat a specific pool using the available chemicals.

The system output is based on multiple input signals. For example, the system may take into account a geographic location of a pool, what the weather is going to be, historical LSI, regular pool LSI readings, trends in the data, chemical preferences/availability, etc., to generate a recommendation. The hardware computing device for recording the dosages corresponds to a client portion of the system architecture. An application server executes logic in view of a database that stores the information, such that a processor can provide a single output recommendation/instruction through the pool care application running on the client device.

Accordingly, the pool care application including the dosage calculation application provides skilled pool care capabilities to lay people who are caring for a swimming pool. That is, pool people are able to properly care for swimming pools using the pool care application/dosage calculation application even when the pool people do not have a background in water chemistry, as the dosage calculation application indicates specific measures for a pool person to perform for a specific pool. For example, the dosage calculation application may indicate what steps the pool person should take when the water temperature is 74 degrees F. versus 76 degrees F. The dosage calculation application outputs the indication based on not only the input values and/or modified values, but also based on client preferences, activity, the weather, geographic region of the swimming pool, and the like.

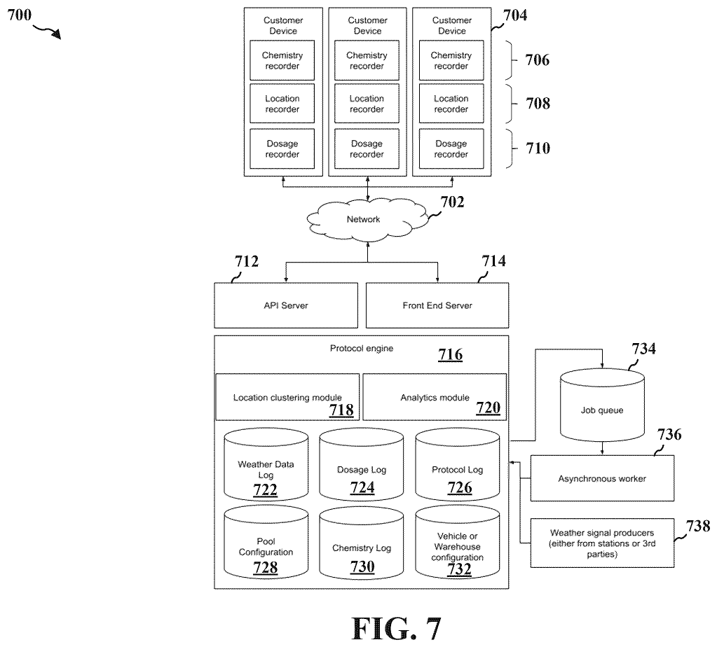

FIG. 7 is a block diagram 700 illustrating a system architecture for generating a pool care recommendation. The system architecture may be a networked architecture. For example, the system architecture may comprise client-side components and server-side components on opposite sides of a network 702.

The client-side of the network 702 includes one or more customer devices 704. The customer devices 704 may be smartphones, electronic tablets, laptop computers, desktop computers, or any other type of computing device configured to execute the pool care application. The customer devices 704 illustrated in the diagram 700 can be the same or different types of customer devices 704.

Each customer device 704 may include a chemistry recorder 706, a location recorder 708, and/or a dosage recorder 710. The chemistry recorder 706 records the water chemistry of a pool, such as for different measurement recordation instances. The chemistry recorder 706 causes the water chemistry measurements to be communicated over the network 702 and stored in a chemistry log 730 of a protocol engine 716. The location recorder 708 records a location of a subject swimming pool. The location may be a geographic location or region, address information, elevation information, coordinates, or the like. The location recorder 708 causes location information to be communicated over the network 702 and received by a location clustering module 718 that facilitates pool care recommendations based on the location information. The dosage recorder 710 records applied chemical dosages to the pool water, such as for different dosage application instances. The dosage recorder 710 causes dosage information to be communicated over the network 702 and stored in a dosage log 724 of the protocol engine 716.

The server-side of the network 702 includes an API server 712 and a front-end server 714 in addition to the protocol engine 716. The API server 712 enables integration between the client-side components of the network 702 and the server-side components of the network 702, such that different software systems may communicate with each other. For example, the API server 712 may enable communication of the chemistry information, location information, and/or dosage information from the pool care application executing on the customer devices 704 to the protocol engine 716 on the server-side of the network 702. The API server 712 may also enable communication and functionality between client-side components of the network 702 and third-party services. The front-end server 714 can be implemented to serve content and handle user interactions related to a presentation layer of an application, such as the pool care application. For example, the front-end server 714 may be an application server that is responsible for handling user interactions, causing a UI to be displayed at the customer devices 704, and/or processing user requests.

The protocol engine 716 may comprise a plurality of databases, including a weather data log 722, the dosage log 724, a protocol log 726, a pool configuration 728, the chemistry log 730, and/or a vehicle or warehouse configuration 732. As described above, the dosage log 724 includes the dosage information (both current and historical) received from the dosage recorder 710 and the chemistry log 730 includes water chemistry information (both current and historical) received from the chemistry recorder 706. The weather data log 722 may include historical weather data associated with specific pools, such that environmental/weather conditions may be correlated with the times of different water chemistry readings. The protocol log 726 stores protocols to be followed for different pool conditions. The pool configuration 728 stores pool-specific information for a pool owner's swimming pool. The vehicle or warehouse configuration 732 stores information about pool service vehicles (e.g., location information of a pool service professional) and information about chemical supply warehouses (e.g., which warehouse has a chemical in stock, an available amount of the chemical, a price of the chemical, etc.).

The protocol engine 716 further includes the location clustering module 718 and an analytics module 720. The location clustering module 718 may be configured to group pools into a cluster when multiple pools share common characteristics. For instance, multiple pools being located within a certain distance of each other may be an example of one criterion that can be used for grouping the multiple pools into the cluster. Other common characteristics among the multiple pools can also be identified for grouping or clustering the pools in different ways. In this manner, the system can output a recommendation on a pool cluster basis, as opposed to generating an explicit recommendation for each and every pool being hosted by the system. The analytics module 720 provides data analytics indicative of how a particular pool has been cared for over time. The analytics module 720 may access information from one or more of the databases 722-732 to generate and output the data analytics.

The protocol engine 716 may also be in communication with a job queue database 734, an asynchronous worker device 736, and/or devices of weather signal producers 738 such as weather stations or third-party providers. The job queue 734 stores a list of jobs to which a pool service professional is to be dispatched. When a new job is received, such as from a client device 704 via the server-side components, the pool service professional may add the new job to the job queue 734. Similarly, when a job is complete, the pool service professional may remove the job from the job queue 734. An asynchronous worker device 736 of the pool service professional may be used to adjust the job queue 734, receive job queue information, and generally interact with the customer devices 704 and the server-side components of the system. In some examples, weather signal producers 738, such as weather stations or third-part providers, provide weather forecast information to the protocol engine 716, which bases a generated recommendation on the weather forecast information.

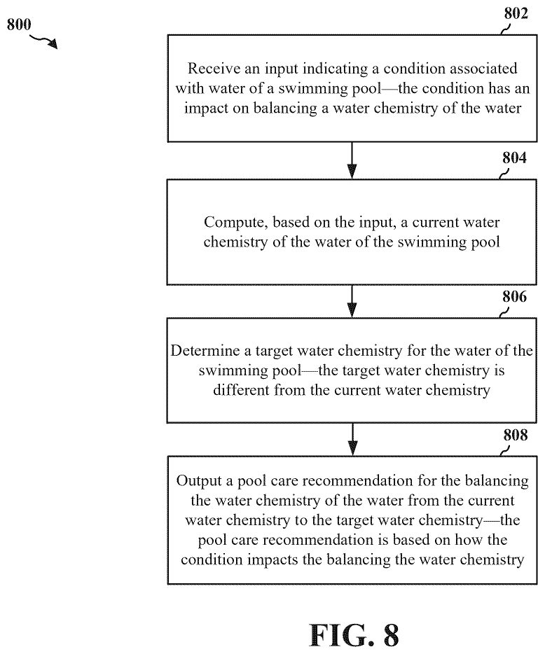

FIG. 8 is flowchart 800 of a method of processing pool care information. The method may be performed by processing logic that may comprise hardware (e.g., circuitry, dedicated logic, programmable logic, a processor, a processing device, a CPU, a system-on-chip, etc.), software (e.g., instructions running/executing on a processing device), firmware (e.g., microcode), or a combination thereof. In some embodiments, at least a portion of the method may be performed based on aspects of FIGS. 1-7.

With reference to FIG. 8, the method illustrates example functions used by various embodiments. Although specific function blocks (“blocks”) are disclosed in the method, such blocks are examples. That is, embodiments are well suited to performing various other blocks or variations of the blocks recited in the method. It is appreciated that the blocks in the method may be performed in an order different than presented, and that not all of the blocks in the method may be performed.

The method begins at block 802, where processing logic receives an input indicating a condition associated with water of a swimming pool—the condition has an impact on balancing a water chemistry of the water.

At block 804, the processing logic computes, based on the input, a current water chemistry of the water of the swimming pool.

At block 806, the processing logic determines a target water chemistry for the water of the swimming pool—the target water chemistry is different from the current water chemistry.

At block 808, the processing logic outputs a pool care recommendation for the balancing the water chemistry of the water from the current water chemistry to the target water chemistry—the pool care recommendation is based on how the condition impacts the balancing the water chemistry.

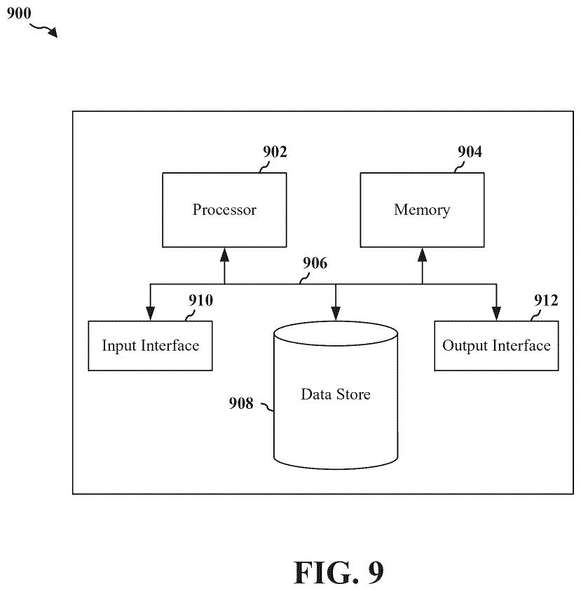

FIG. 9 is a high-level illustration of an exemplary computing device 900 that can be used in accordance with the systems and methodologies disclosed herein. For instance, the computing device 900 may be or include the device 104. The computing device 900 includes at least one processor 902 that executes instructions that are stored in a memory 904. The instructions may be, for instance, instructions for implementing functionality described as being carried out by one or more modules or instructions for implementing one or more of the methods described above. The processor 902 may access the memory 904 by way of a system bus 906.

The computing device 900 additionally includes a data store 908 that is accessible by the processor 902 by way of the system bus 906. The data store 908 may include executable instructions and the like. The computing device 900 also includes an input interface 910 that allows external devices to communicate with the computing device 900. For instance, the input interface 910 may be used to receive instructions from an external computing device, from a user, etc. The computing device 900 also includes an output interface 912 that interfaces the computing device 900 with one or more external devices.

Additionally, while illustrated as a single system, it is to be understood that the computing device 900 may be a distributed system. Thus, for instance, several devices may be in communication by way of a network connection and may collectively perform tasks described as being performed by the computing device 900.

The description herein is provided to enable a person skilled in the art to practice the various aspects described herein. Various modifications to these aspects will be readily apparent to those skilled in the art, and the generic principles defined herein may be applied to other aspects. Thus, the claims are not limited to the aspects described herein but are to be interpreted in view of the full scope of the present disclosure consistent with the language of the claims.

Reference to an element in the singular does not mean “one and only one” unless specifically stated, but rather “one or more.” Terms such as “if,” “when,” and “while” do not imply an immediate temporal relationship or reaction. That is, these phrases, e.g., “when,” do not imply an immediate action in response to or during the occurrence of an action, but simply imply that if a condition is met then an action will occur, but without requiring a specific or immediate time constraint for the action to occur. Unless specifically stated otherwise, the term “some” refers to one or more. Combinations such as “at least one of A, B, or C” or “one or more of A, B, or C” include any combination of A, B, and/or C, such as A and B, A and C, B and C, or A and B and C, and may include multiples of A, multiples of B, and/or multiples of C, or may include A only, B only, or C only. Sets should be interpreted as a set of elements where the elements number one or more.

Unless otherwise specifically indicated, ordinal terms such as “first” and “second” do not necessarily imply an order in time, sequence, numerical value, etc., but are used to distinguish between different instances of a term or phrase that follows each ordinal term.

Structural and functional equivalents to elements of the various aspects described throughout this disclosure that are known or later come to be known to those of ordinary skill in the art are expressly incorporated herein by reference and are encompassed by the claims. The words “module,” “mechanism,” “element,” “device,” and the like may not be a substitute for the word “means.” As such, no claim element is to be construed as a means plus function unless the element is expressly recited using the phrase “means for.” As used herein, the phrase “based on” shall not be construed as a reference to a closed set of information, one or more conditions, one or more factors, or the like. In other words, the phrase “based on A”, where “A” may be information, a condition, a factor, or the like, shall be construed as “based at least on A” unless specifically recited differently.

The following aspects are illustrative only and may be combined with other aspects or teachings described herein, without limitation.

Example 1 is a method of processing pool care information, including: receiving an input indicating a condition associated with water of a swimming pool, the condition having an impact on balancing a water chemistry of the water; computing, based on the input, a current water chemistry of the water of the swimming pool; determining a target water chemistry for the water of the swimming pool, the target water chemistry being different from the current water chemistry; and outputting, by a processing device, a pool care recommendation for the balancing the water chemistry of the water from the current water chemistry to the target water chemistry, the pool care recommendation being based on how the condition impacts the balancing the water chemistry.

Example 2 may be combined with Example 1 and includes that the condition having the impact on the balancing the water chemistry is at least one of: historical pool care data for the swimming pool, historical dosage data for one or more chemicals, historical LSI values, a trend in historical data associated with the water chemistry, weather information, an environmental condition, location information of the swimming pool, preference information, or chemical supply availability.

Example 3 may be combined with any of Examples 1-2 and includes that the pool care recommendation is a chemical dosage recommendation for dosing the water of the swimming pool based on at least one of: one or more chemical types or a quantity of one or more chemicals.

Example 4 may be combined with any of Examples 1-3 and includes that the pool care recommendation further indicates an amount of time to elapse after the dosing the water of the swimming pool before the water is recommended for swimming.

Example 5 may be combined with any of Examples 1-4 and further includes storing, in a database, a value of the current water chemistry computed based on the input, the value of the current water chemistry being added to historical water chemistry data in the database.

Example 6 may be combined with any of Examples 1-5 and includes that the determining the target water chemistry for the water of the swimming pool is based on at least one of: an indicated water chemistry preference or historical pool care behavior.

Example 7 may be combined with any of Examples 1-6 and further includes generating a notification based on the pool care recommendation, the notification indicating at last one of: a recommended date to perform pool care, a remaining on-site supply of one or more chemicals for dosing the water of the swimming pool, or a supplier of the one or more chemicals.

Example 8 may be combined with any of Examples 1-7 and further includes receiving, from a third-party computing device, a service notification indicating at least one of: the pool care recommendation being added to a job queue database, real-time location information of a pool servicer, or a confirmation that the swimming pool has been serviced.

Example 9 may be combined with any of Examples 1-8 and includes that the outputting the pool care recommendation is based on predictive modeling of the water chemistry of the water.

Example 10 may be combined with any of Examples 1-9 and includes that the condition is one of a plurality of conditions having an impact on the balancing the water chemistry of the water, the pool care recommendation being based on how the plurality of conditions impacts the balancing the water chemistry of the water.

Example 11 is an apparatus for implementing a method as in any of Examples 1-10.

Example 12 is a system for implementing a method as in any of Examples 1-10.

Example 13 is an apparatus including means for implementing a method as in any of Examples 1-10.

Example 14 is a non-transitory computer-readable medium storing computer executable code, the code when executed by at least one processor causes the at least one processor to implement a method as in any of Examples 1-10.

What is claimed is:

1. A method of processing pool care information, comprising:
receiving an input indicating a condition associated with water of a swimming pool, the condition having an impact on balancing a water chemistry of the water;
computing, based on the input, a current water chemistry of the water of the swimming pool;
determining a target water chemistry for the water of the swimming pool, the target water chemistry being different from the current water chemistry; and
outputting, by a processing device, a pool care recommendation for the balancing the water chemistry of the water from the current water chemistry to the target water chemistry, the pool care recommendation being based on how the condition impacts the balancing the water chemistry.

2. The method of claim 1, wherein the condition having the impact on the balancing the water chemistry is at least one of:
historical pool care data for the swimming pool,
historical dosage data for one or more chemicals,
historical Langelier saturation index (LSI) values,
a trend in historical data associated with the water chemistry,
weather information,
an environmental condition,
location information of the swimming pool,
preference information, or
chemical supply availability.

3. The method of claim 1, wherein the pool care recommendation is a chemical dosage recommendation for dosing the water of the swimming pool based on at least one of: one or more chemical types or a quantity of one or more chemicals.

4. The method of claim 3, wherein the pool care recommendation further indicates an amount of time to elapse after the dosing the water of the swimming pool before the water is recommended for swimming.

5. The method of claim 1, further comprising:
storing, in a database, a value of the current water chemistry computed based on the input, the value of the current water chemistry being added to historical water chemistry data in the database.

6. The method of claim 1, wherein the determining the target water chemistry for the water of the swimming pool is based on at least one of: an indicated water chemistry preference or historical pool care behavior.

7. The method of claim 1, further comprising:
generating a notification based on the pool care recommendation, the notification indicating at last one of: a recommended date to perform pool care, a remaining on-site supply of one or more chemicals for dosing the water of the swimming pool, or a supplier of the one or more chemicals.

8. The method of claim 1, further comprising:
receiving, from a third-party computing device, a service notification indicating at least one of: the pool care recommendation being added to a job queue database, real-time location information of a pool servicer, or a confirmation that the swimming pool has been serviced.

9. The method of claim 1, wherein the outputting the pool care recommendation is based on predictive modeling of the water chemistry of the water.

10. The method of claim 1, wherein the condition is one of a plurality of conditions having an impact on the balancing the water chemistry of the water, the pool care recommendation being based on how the plurality of conditions impacts the balancing the water chemistry of the water.

11. An apparatus for processing pool care information, comprising:
a memory; and
at least one processor coupled to the memory and configured to:
receive an input indicating a condition associated with water of a swimming pool, the condition having an impact on balancing a water chemistry of the water;
compute, based on the input, a current water chemistry of the water of the swimming pool;
determine a target water chemistry for the water of the swimming pool, the target water chemistry being different from the current water chemistry; and
output a pool care recommendation for the balancing the water chemistry of the water from the current water chemistry to the target water chemistry, the pool care recommendation being based on how the condition impacts the balancing the water chemistry.

12. The apparatus of claim 11, wherein the condition having the impact on the balancing the water chemistry is at least one of:
historical pool care data for the swimming pool,
historical dosage data for one or more chemicals,
historical Langelier saturation index (LSI) values,
a trend in historical data associated with the water chemistry,
weather information,
an environmental condition,
location information of the swimming pool,
preference information, or
chemical supply availability.

13. The apparatus of claim 11, wherein the pool care recommendation is a chemical dosage recommendation for dosing the water of the swimming pool based on at least one of: one or more chemical types or a quantity of one or more chemicals.

14. The apparatus of claim 11, wherein the at least one processor is further configured to:
store, in a database, for a value of the current water chemistry computed based on the input, the value of the current water chemistry being added to historical water chemistry data in the database.

15. The apparatus of claim 11, wherein the determination of the target water chemistry for the water of the swimming pool is based on at least one of: an indicated water chemistry preference or historical pool care behavior.

16. The apparatus of claim 11, wherein the at least one processor is further configured to:
generate a notification based on the pool care recommendation, the notification indicating at last one of: a recommended date to perform pool care, a remaining on-site supply of one or more chemicals for dosing the water of the swimming pool, or a supplier of the one or more chemicals.

17. The apparatus of claim 11, wherein the at least one processor is further configured to:
receive, from a third-party computing device, a service notification indicating at least one of: the pool care recommendation being added to a job queue database, real-time location information of a pool servicer, or a confirmation that the swimming pool has been serviced.

18. The apparatus of claim 11, wherein the output for the pool care recommendation is based on predictive modeling of the water chemistry of the water.

19. The apparatus of claim 11, wherein the condition is one of a plurality of conditions having an impact on the balancing the water chemistry of the water, the pool care recommendation being based on how the plurality of conditions impacts the balancing the water chemistry of the water.

20. A non-transitory computer-readable medium storing computer executable code, the code when executed by at least one processor causes the at least one processor to:
receive an input indicating a condition associated with water of a swimming pool, the condition having an impact on balancing a water chemistry of the water;
compute, based on the input, a current water chemistry of the water of the swimming pool;
determine a target water chemistry for the water of the swimming pool, the target water chemistry being different from the current water chemistry; and
output, by a processing device, a pool care recommendation for the balancing the water chemistry of the water from the current water chemistry to the target water chemistry, the pool care recommendation being based on how the condition impacts the balancing the water chemistry.
